(19) United States
(12) Patent Application Publication
(10) Pub. No.: US 2023/0285182 A1
(43) Pub. Date: Sep. 14, 2023

(54) Flexible ball mouth guard for the prevention of teeth grinding, jaw clenching and TMJ

(71) Applicant: Karen Q. Roth, Hillsdale, NJ (US)

(72) Inventor: Karen Q. Roth, Hillsdale, NJ (US)

(21) Appl. No.: 18/445,009

(22) Filed: Feb. 28, 2023

Related U.S. Application Data
(60) Provisional application No. 63/372,400, filed on Mar. 9, 2022.

(51) Int. Cl. A61F 5/56

(52) U.S. Cl. CPC A61F 5/566; A61F 2005/563

(57) ABSTRACT

The new and improved mouth guard of the invention comprises a soft flexible mouth guard preferably of flexible polymer, rubber or plastic materials with a channel for one's teeth and a flexible ball filled with a flexible material that is attached to the exterior base of the mouth guard tray. The flexible ball is positioned under the upper two to four front teeth or above the lower two to four front teeth and extends outwardly to provide a flexible barrier between the upper and lower front teeth for the purpose of absorbing tension and pressure when biting and to prevent contact of the poster teeth thus preventing grinding, clenching and dental issues. Both the mouth guard and flexible ball can vary in size and shape.

### CROSS-REFERENCE TO RELATED APPLICATIONS

This application claims the benefit of U.S. Provisional Application No. 63/372,400 filed Mar. 9, 2022. The provisional application is entitled Springy Ball Mouth Guard for the Prevention of Teeth Grinding, Jaw Clenching and TMJ

### STATEMENT REGARDING FEDERALLY SPONSORED RESEARCH OR DEVELOPMENT THE NAMES OF THE PARTIES TO A JOINT RESEARCH AGREEMENT

### N/A

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

N/A

### STATEMENT REGARDING PRIOR DISCLOSURES BY THE INVENTOR OR A JOINT INVENTOR

N/A

### BACKGROUND OF THE INVENTION

This invention is intended to alleviate and possibly eliminate the problems associated with teeth grinding, clenching and TMJ as discussed in applicant's U.S. Pat. No. 11,564,828 B2. Bruxism is excessive teeth grinding and/or jaw clenching and is recognized as a serious problem which can lead to more serious health issues. Providing a mouth guard to prevent bruxism will assist in preventing damage to the teeth and other oral health complications that could arise. It is also important to provide a mouth guard to protect teeth from injury and trauma while participating in sports. Having a customized mouth guard with an integral flexible ball filled with flexible material will assist in preventing teeth grinding, clenching, TMJ and dental issues.

### (1) FIELD OF THE INVENTION

This invention relates to the field of mouth guards and in particular to devices for preventing teeth grinding, clenching, TMJ and related dental issues.

### (2) DESCRIPTION OF RELATED ART INCLUDING INFORMATION DISCLOSED UNDER 37 CFR 1.97 AND 1.98

The prior art includes the following patents:
U.S. Pat. No. 11,564,828 by applicant discloses a mouth guard to prevent teeth grinding, jaw clenching and TMJ. The mouth guard discloses a bubble or air-filled spherical cavity that is positioned around and under the upper front teeth or around and above the lower front teeth to prevent the posterior teeth from touching thus preventing clenching and grinding.
U.S. Pat. No. 9,681,978 by applicant discloses a mouth guard for the prevention of jaw clenching and teeth grinding. The mouth guard discloses a tab disposed between a person's top and bottom front teeth to resist full closure of the jaws so the upper and lower back teeth do not engage each other.
U.S. Pat. No. 6,675,806 to Kittelsen, et al, discloses a composite force absorbing mouth guard with elastomeric traction pads on the bottom of the mouth guard. A hard reverse bite plate wedge is thicker towards the rear of thereof to place the lower jaw in an optimum position to prevent nerve impingement as well as spacing the upper and lower jaws apart.
U.S. Pat. No. 8,205,618 to Berghash discloses a sports mouth guard of a three laminate construction. The mouth guard includes and upper portion with holes in a channel floor and lower platform with posts aligned to fit into the holes providing a protective cushion.
U.S. Pat. No. 9,968,419 to Alverez, et al, discloses an appliance for preventing bruxism with protuberances and also for improving air intake.

The relevant prior art noted above does not disclose or anticipate the Flexible Ball filled with flexible material mouth guard feature of applicant's invention.

### BRIEF SUMMARY OF THE INVENTION

The present invention relates to mouth guards and in particular to a soft flexible elastic mouth guard of various materials such as a flexible polymer, silicone rubber, an ultra-low-density polyethylene, or plastic designed to cover and protect a person's upper or lower teeth. The mouth guard also includes a flexible ball filled with flexible material which is attached to the exterior base of the mouth guard tray and extends outwardly from the upper or lower exterior base of the mouth guard tray. The flexible ball is positioned under the upper two to four front teeth or above the lower two to four front teeth to absorb tension and pressure when biting and to prevent contact of the upper and lower posterior teeth thus preventing clenching and grinding.

Accordingly, it is an object of this invention to provide a new and improved mouth guard to eliminate the problems associated with teeth grinding, clenching and TMJ.

It is another object of this invention to provide a new and improved form fitting mouth guard adapted to cover the upper or lower front teeth or full set of teeth to combat bruxism and TMJ.

It is a more specific object of this invention to provide a new and improved easy to manufacture mouth guard having a portion with a flexible ball filled with flexible material that is attached to the exterior base of the upper or lower mouth guard tray and positioned below or above the front teeth and extending outwardly from the mouth guard tray to provide a flexible barrier between the upper and lower front teeth to prevent grinding, clenching and TIM.

It is a further object of this invention to provide a new and improved mouth guard to prevent bruxism or trauma having a plurality of spaced flexible balls filled with flexible material that are attached to the exterior base of the mouth guard tray and extending outwardly from the mouth guard tray to engage the opposite set of teeth and provide a greater flexible barrier between the upper and lower teeth absorbing shock and tension when biting.

### BRIEF DESCRIPTION OF THE SEVERAL VIEWS OF THE DRAWING(S)

The above and other objects of this invention may be more clearly seen when viewed in conjunction with the accompanying drawings wherein:

FIG. 1 shows a perspective view of the mouth guard with flexible ball filled with flexible material attached to the exterior base of the mouth guard under the upper two to four front teeth in a mouth;

FIG. 2 shows the mouth guard with flexible ball filled with flexible material attached to the exterior base of the mouth guard above the lower two to four front teeth in a perspective view in a mouth;

FIG. 3 shows a perspective view of the mouth guard worn over the upper two to four front teeth with flexible ball filled with flexible material attached to the exterior base of the mouth guard;

FIG. 4 shows the mouth guard worn over the lower two to four front teeth with flexible ball filled with flexible material attached to the exterior base of the mouth guard in a perspective view;

FIG. 5 shows a perspective view of the mouth guard with flexible ball filled with flexible material;

FIG. 6 shows a front view of the mouth guard with flexible ball filled with flexible material thereof.

### DETAILED DESCRIPTION OF THE INVENTION

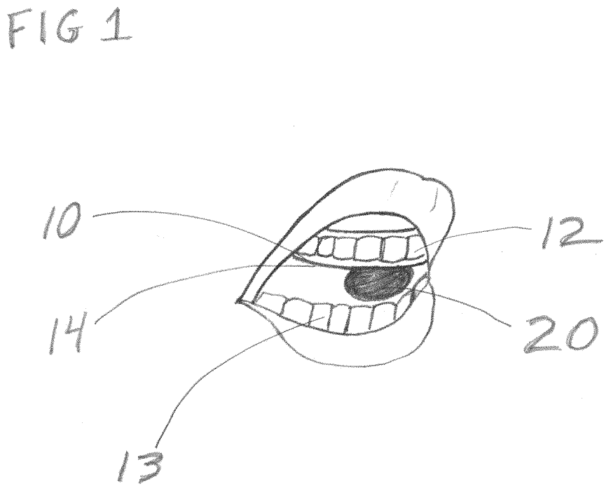

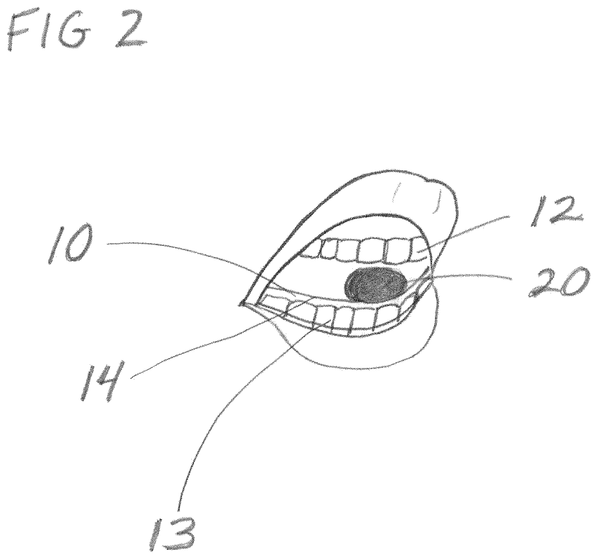

Referring now to the drawings and particularly FIGS. 1 and 2, the invention comprises a mouth guard 10 which can be worn day or night to prevent teeth grinding, clenching and TMJ. The mouth guard 10 is sized and configured in a substantially U-shape tray having a channel 11 to encompass a person's upper 12 or lower 13 set of teeth. The mouth guard 10 further includes an integral flexible ball filled with flexible material 20 on the exterior base 14 of the mouth guard U-shape where the front teeth are located. The integral design of the flexible ball 20 makes the mouth guard 10 easy to manufacture compared with mouth guards having tabs or air-filled cavity protruding from the interior tray of the channel and around the front teeth.

The flexible ball filled with flexible material extends outwardly from the upper or lower exterior base 14 of the mouth guard 10 depending on whether it is affixed to the upper 12 or lower set of teeth 13. The flexible ball 20 can be placed under the upper two to four front teeth as shown in FIG. 1. Alternatively, the mouth guard 10 may be positioned on the lower set of teeth 13 and the flexible ball 20 can be positioned above the lower two to four front teeth as shown in FIG. 2.

The purpose of the mouth guard 10 with flexible ball filled with flexible material 20 positioned under the upper 12 or above the lower 13 front teeth is to provide a flexible barrier between the upper and lower front teeth to absorb tension and pressure when biting and to prevent contact of the upper and lower posterior teeth thus preventing grinding and clenching of posterior teeth.

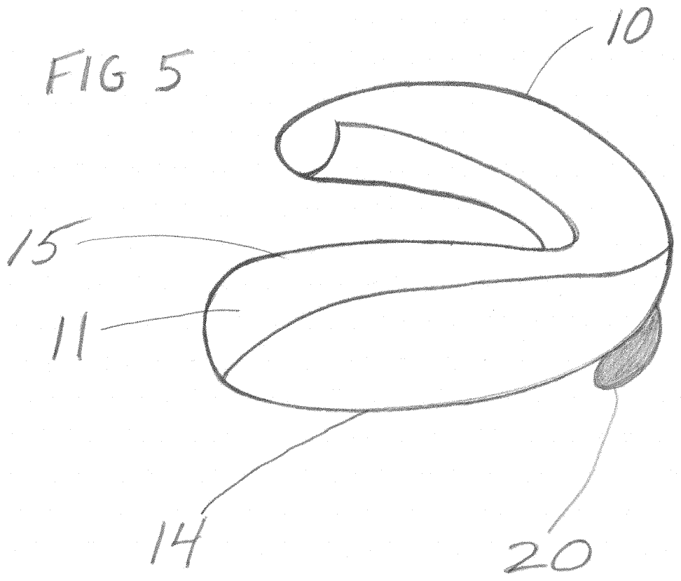

FIG. 5 is a perspective view of the mouth guard 10 thereof showing the tray 15 which is a flexible elastic material that molds to an individual's teeth which are positioned in the channel 11. The flexible ball 20 extending from the exterior base 14 at the U shape of the tray is positioned at the front of the mouth guard 10 where it engages the opposite teeth.

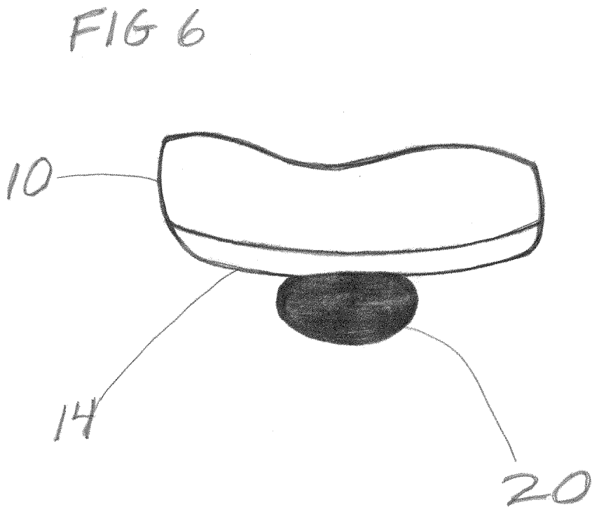

FIG. 6 is a frontal view of the mouth guard 10 that prominently displays the flexible ball filled with flexible material 20 attached to the exterior base 14 of the mouth guard extending downwardly and outwardly both anteriorly and posteriorly from the mouth guard tray.

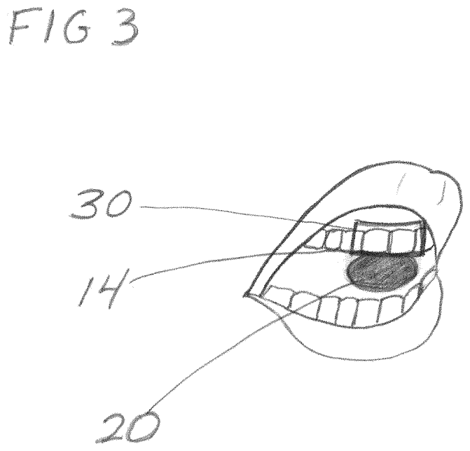

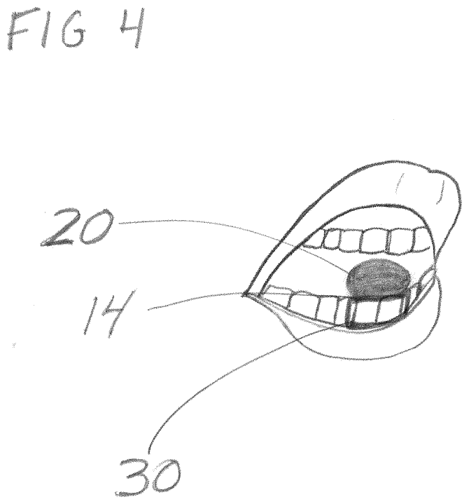

In alternative embodiments, as shown in FIGS. 3 and 4, the mouth guard is not limited to encompass a person's upper 12 or lower 13 set of teeth, it can be adjusted in size to comprise a shorter tray and channel adapted to fit over a person's upper 12 or lower 13 two to four front teeth. This smaller mouth guard is affixed to the upper two to four front teeth 30 with flexible ball filled with flexible material 20 attached to the exterior base 14 of the mouth guard tray 15 and positioned under the upper two to four front teeth extending downwardly and outwardly both anteriorly and posteriorly from the exterior base of the mouth guard tray, see FIG. 3. Alternatively, this smaller mouth guard is affixed to the lower two to four front teeth 30 with flexible ball filled with flexible material 20 attached to the exterior base 14 of the mouth guard tray 15 and positioned above the lower two to four front teeth extending upwardly and outwardly both anteriorly and posteriorly from the exterior base of the mouth guard tray, see FIG. 4.

This alternative embodiment, a smaller mouth guard having a shorter tray and channel in FIGS. 3 and 4 adapted to fit over a person's upper or lower two to four front teeth may be more beneficial to an individual's dental issues or needs as opposed to a mouth guard adapted to fit over a person's upper or lower set of teeth. The flexible ball can range in size, shape and thickness. The mouth guard 10 or 30 and the attached flexible ball filled with flexible material can be made of a semi-rigid or flexible polymer, silicone rubber, and ultra-low-density polyethylene or a variety of plastic materials and can be adjusted or molded to one's mouth. The purpose of the mouth guard 30 with flexible ball filled with flexible material is to create a flexible barrier between the upper 12 and lower 13 two to four front teeth to absorb pressure and tension when biting and to prevent contact of the upper and lower posterior teeth and assist in preventing grinding, clenching and TMJ.

1. A mouth guard adapted to prevent teeth grinding and clenching of upper and lower posterior teeth by providing a flexible barrier between the upper two to four front teeth and the lower two to four front teeth which prevents contact of posterior teeth comprises:
A tray having a substantially U-shaped channel including opposing spaced side walls and a base having a thickness adapted to fit over the upper teeth and mold to their configuration; and,
A flexible ball filled with flexible material attached to exterior base of the mouth guard tray positioned under the upper two to four front teeth and extending downwardly and outwardly both anteriorly and posteriorly to provide a flexible barrier between the upper and lower front teeth and provide spacing between the upper and lower posterior teeth to prevent teeth grinding and clenching of the posterior teeth.

2. A mouth guard in accordance with claim 1 wherein:
the mouth guard tray comprises a soft flexible polymer, silicone rubber, ultra-low-polyethylene or a variety of plastic materials adapted to mold to an individual's teeth and the flexible ball filled with a flexible material is attached to the exterior base of the mouth guard tray.

3. A mouth guard in accordance with claim 2 wherein:
the tray material is a flexible polymer, silicone rubber, an ultra-low-density polyethylene or a variety of plastic materials and the flexible ball portion includes a ball filled with a flexible polymer, silicone rubber, an ultra-low-density polyethylene or a variety of plastic materials.

4. A mouth guard adapted to prevent teeth grinding and clenching of the upper and lower posterior teeth by providing a flexible barrier between the upper two to four front teeth and the lower two to four front teeth which prevents contact of posterior teeth comprises:
A tray having a substantially U-shaped channel including opposing spaced side walls and a base having a thickness adapted to fit over the lower teeth and mold to their configuration; and,
A flexible ball filled with a flexible material attached to the exterior base of the mouth guard tray positioned above the lower two to four front teeth and extending upwardly and outwardly both anteriorly and posteriorly to provide a flexible barrier between the upper and lower front teeth and provide spacing between the upper and lower posterior teeth to prevent teeth grinding and clenching of the posterior teeth.

5. A mouth guard adapted to prevent teeth grinding and clenching of the upper and lower posterior teeth by providing a flexible barrier between the upper two to four front teeth and the lower two to four front teeth which prevents contact of posterior teeth comprises:
A tray having a U-shaped channel including opposing spaced side walls and a base having a thickness adapted to fit over the upper or lower two to four front teeth and mold to their configuration. and,
A flexible ball filled with a flexible material attached to the exterior base of the mouth guard tray positioned below the upper two to four front teeth and extending downwardly and outwardly both anteriorly and posteriorly or positioned above the lower two to four front teeth and extending upwardly and outwardly both anteriorly and posteriorly to provide a flexible barrier between the upper and lower front teeth and provide spacing between the upper and lower posterior teeth to prevent teeth grinding and clenching of the posterior teeth.
